(19) United States
(12) Patent Application Publication
(10) Pub. No.: US 2002/0015718 A1
(43) Pub. Date: Feb. 7, 2002

(54) Use of physiologically compatible sulfinic acids as antioxidant or free-radical scavenger in cosmetic or dermatological preparations

(76) Inventors: Inge Kruse, Hamburg (DE); Heiner Max, Hamburg (DE); Birgit Hargens, Hamburg (DE); Uwe Schonrock, Nahe (DE); Claudia Hinze, Aachen (DE)

Correspondence Address: Howard C. Lee, Norris McLaughlin & Marcus, 30th Floor, 220 East 42nd Street, New York, NY, 10017, US

(21) Appl. No.: 09/894,619

(22) Filed: Jun. 28, 2001

(30) Foreign Application Priority Data
Jul. 1, 2000 (DE) 10032165.8

(51) Int. Cl. A61K 7/00; A61K 31/185

(52) U.S. Cl. 424/401; 514/553

(57) ABSTRACT

Cosmetic or dermatological preparations which have a pH of >5, with a content of physiologically compatible sulfinic acids.

The present invention relates to cosmetic and dermatological preparations with an effective content of physiologically compatible sulfinic acids. In particular, the present invention relates to cosmetic preparations with effective protection against harmful oxidation processes in the skin, but also the protection of cosmetic preparations themselves and/or the protection of the constituents of cosmetic preparations against harmful oxidation processes.

The present invention further relates to antioxidants, preferably those which are used in skincare cosmetic or dermatological preparations. In particular, the invention also relates to cosmetic and dermatological preparations comprising such antioxidants. In a preferred embodiment, the present invention relates to cosmetic and dermatological preparations for the prophylaxis and treatment of cosmetic or dermatological skin changes, such as, for example, skin aging, in particular skin aging caused by oxidative processes.

The present invention further relates to active ingredients and preparations comprising such active ingredients for the cosmetic and dermatological treatment or prophylaxis of erythematous, inflammatory, allergic or autoimmune-reactive symptoms, in particular dermatoses.

The present invention further relates to active ingredient combinations and preparations used for the prophylaxis and treatment of light-sensitive skin, in particular of photodermatoses.

The harmful effect of the ultraviolet part of solar radiation on the skin is generally known. While rays having a wavelength of less than 290 nm (the “UVC region”) are absorbed by the ozone layer in the earth's atmosphere, rays in the range between 290 nm and 320 nm, the “UVB region”, cause erythema, simple sunburn or even burns of greater or lesser severity.

The erythema activity maximum of sunlight is stated as the narrower range around 308 nm.

Numerous compounds are known for protecting against UVB radiation; these are usually derivatives of 3-benzylidenecamphor, 4-aminobenzoic acid, of cinnamic acid, of salicylic acid, of benzophenone and also of 2-phenylbenzimidazole.

For the range between about 320 nm and about 400 nm, of the “UVA region”, it is also important to have available filter substances, since the rays of that region can also cause reactions in the case of light-sensitive skin. It has been found that UVA radiation leads to damage of the elastic and collagenous fibres of connective tissue, causing premature aging of the skin, and that it is to be regarded as a cause of numerous phototoxic and photoallergic reactions. The harmful effect of UVB radiation can be intensified by UVA radiation.

To protect against the rays of the UVA region, therefore, certain derivatives of dibenzoylmethane are used, the photostability of which is insufficient (Int. J. Cosm. Science 10, 53 (1988)).

However, UV radiation can also lead to photochemical reactions, in which case the photochemical reaction products intervene in the skin's metabolism.

Such photochemical reaction products are predominantly free-radical compounds, for example hydroxyl radicals. Undefined free-radical photoproducts which form in the skin itself can also display uncontrolled secondary reactions because of their high reactivity. However, singlet oxygen, a non-radical excited state of the oxygen molecule, can also be formed during UV irradiation, as can short-lived epoxides and many others. Singlet oxygen, for example, differs from the normal triplet oxygen (free-radical ground state) by virtue of its increased reactivity. However, excited, reactive (free-radical) triplet states of the oxygen molecule also exist.

Furthermore, UV radiation is a type of ionizing radiation. There is therefore the risk that UV exposure may also produce ionic species, which then, for their part, are capable of oxidative intervention in the biochemical processes.

In order to avoid these reactions, it is possible to incorporate additional antioxidants and/or free-radical scavengers into the cosmetic or dermatological formulations.

It has already been proposed to use vitamin E, a substance with known antioxidative action in light protection formulations, the effect achieved here nevertheless falls a long way short of expectations here as well.

The object of the invention was therefore also to provide cosmetic, dermatological and pharmaceutical active ingredients and preparations and light protection formulations which are used for the prophylaxis and treatment of light-sensitive skin, in particular photodermatoses, preferably PLD.

Other names for polymorphous light dermatosis are PLD, PLE, Mallorca acne and a large number of other names, as given in the literature (e.g. A. Voelckel et al, Zentralblatt Haut- und Geschlechtskrankheiten (1989), 156, p.2).

Erythematous skin symptoms also occur as accompanying symptoms in certain skin diseases or irregularities. For example, the typical skin rash symptom of acne is generally red to a greater or lesser extent.

Antioxidants are mainly used as substances which protect against spoilage of the preparations in which they are present. Nevertheless, it is known that in human and animal skin as well, undesired oxidation processes may occur. Such processes play an important role in skin aging.

The essay “Skin Diseases Associated with Oxidative Injury” in “Oxidative Stress in Dermatology”, p. 323 ff. (Marcel Decker Inc., New York, Basel, Hong Kong, Editor: Jürgen Fuchs, Frankfurt, and Lester Packer, Berkeley/Calif.) discusses oxidative skin damage and its more obvious causes.

If the aim is to permanently color human hair, only oxidizing hair coloring methods are suitable in practice. During oxidative hair coloring, dye chromophores form as a result of the reaction of precursors (phenols, aminophenols, more rarely also diamines) and bases (in most cases p-phenylenediamine) with the oxidizing agent, in most cases hydrogen peroxide. Hydrogen peroxide concentrations around 6% are normally used for this purpose.

It is usually assumed that in addition to the coloring action, a bleaching action as a result of the hydrogen peroxide also takes place. In oxidatively colored human hair, as in bleached hair, it is possible to detect microscopic holes at sites where melanin granules were present.

The fact is that the oxidizing agent hydrogen peroxide reacts not only with the dye precursors, but also with the hair substance, and in some instances can cause hair damage.

Antioxidants are substances which prevent oxidation processes or which prevent the autoxidation of fats containing unsaturated compounds. Antioxidants, which are also used in the fields of cosmetics and pharmaceuticals, are, for example, α-tocopherol, in particular in the form of the α-tocopheryl acetate, sesamol, gallic acid derivatives, butylhydroxyanisole and butylhydroxytoluene.

Also for reasons of preventing such reactions, it is possible to additionally incorporate antioxidants and/or free-radical scavengers into cosmetic formulations.

Admittedly a number of antioxidants and free-radical scavengers are known. For example U.S. Pat. Nos. 4,144,325 and 4,248,861 and a large number of other documents have already proposed using vitamin E, a substance with known antioxidative action in light protection formulations, although the effect achieved falls a long way short of expectations here as well.

One object of the present invention was to overcome the disadvantages of the prior art. In particular, the aim was to provide active ingredients and/or preparations comprising such active ingredients, the use of which is at least able to reduce, if not completely prevent, damage to skin and/or hair by oxidative effects.

A further object of the present invention was to provide cosmetic preparations which, before or after treatment of the hair with hair coloring preparations, even those with a content of strong oxidizing agents such as, for example, hydrogen peroxide, counter the damaging oxidation effect thereof.

In particular, the aim was to provide active ingredients and preparations comprising such active ingredients for the cosmetic and dermatological treatment and/or prophylaxis of erythematous, inflammatory, allergic or autoimmune-reactive symptoms, in particular dermatoses, but also the symptom of “stinging”.

Finally, it was an object of the present invention to provide antioxidants which are stable over a wide pH range, in particular in the pH range >5.

It was therefore surprising and could not have been foreseen by the person skilled in the art that cosmetic or dermatological preparations which have a pH of >5, with a content of physiologically compatible sulfinic acids, and the use of physiologically compatible sulfinic acids as antioxidant and/or as free-radical scavenger in cosmetic or dermatological preparations which have a pH of >5 achieve these objects and overcome the disadvantages of the prior art.

Although JP-1993-0139844 describes the use of hypotaurine as antioxidant in cosmetic or dermatological preparation, it was not able to give any indication as to the advantageous properties at a high pH.

For the purposes of the present invention, cosmetically compatible sulfinic acids are preferably distinguished by the structure

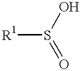

in which R¹ is a branched or unbranched saturated hydrocarbon chain having up to 18 carbon atoms which is unsubstituted or substituted by up to three each of OH groups and/or NH₂ groups.

R¹ is preferably a branched or unbranched saturated hydrocarbon chain having up to 18 carbon atoms which is terminally substituted by a NH₂ group, according to the formula

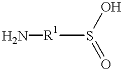

Particular preference is given to 2-aminoethanesulfinic acid (hypotaurine), which is distinguished by the structure

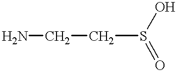

or its betaine form

[chemical structure]

It could not therefore have been foreseen by the person skilled in the art that the physiologically compatible sulfinic acids used according to the invention and cosmetic or dermatological preparations comprising such acids

would be stable and active at a pH >5

are more effective antioxidants

are more effective free-radical scavengers

better prevent the binding of harmful photoproducts to lipids, DNA and proteins

are more effective against skin aging

better protect the skin against photoreactions

would better prevent inflammatory reactions

than the active ingredients, active ingredient combinations and preparations of the prior art. In addition, it could not have been foreseen that in cosmetic or dermatological preparations sulfinic acids are more stable than comparable active ingredients, for example than vitamin C.

The invention therefore relates to the use of physiologically compatible sulfinic acids as antioxidant and also to its use for the treatment and/or prophylaxis of skin aging caused as a result of oxidative stress, and of inflammatory reactions.

The cosmetic or dermatological formulations according to the invention can have the customary composition and can be used for the treatment, care and cleansing of the skin and/or the hair and as a make-up product in decorative cosmetics. They preferably comprise from 0.01% by weight to 10% by weight, preferably from 0.05% by weight to 5% by weight, in particular 0.1-2.0% by weight, based on the total weight of the composition, of one or more physiologically compatible sulfinic acids

For use, the cosmetic and dermatological preparations according to the invention are applied to the skin and/or the hair in an adequate amount in the customary manner for cosmetics.

Cosmetic and dermatological preparations according to the invention can be in various forms. Thus, they can, for example, be a solution, a water-free preparation, an emulsion or microemulsion of the water-in-oil (W/O) type or of the oil-in-water (O/W) type, a multiple emulsion, for example of the water-in-oil-in-water (W/O/W) type, a gel, a solid stick, an ointment or else an aerosol. It is also advantageous to administer physiologically compatible sulfinic acids in encapsulated form, for example in collagen matrices and other customary encapsulation materials, for example as cellulose capsules, in gelatin, wax matrices or liposomally encapsulated. In particular, wax matrices, as are described in DE-A 43 08 282, have proven to be favorable.

For the purposes of the present invention, it is also possible and advantageous to incorporate physiologically compatible sulfinic acids in encapsulated form into aqueous systems or surfactant preparations for cleansing the skin and the hair.

An advantageous embodiment of the present invention is therefore also to be regarded as the use of physiologically compatible sulfinic acids for protecting the skin and/or the hair against oxidative stress, in particular this use of the physiologically compatible sulfinic acids in shampoos and washing formulations.

The cosmetic and dermatological preparations according to the invention, can comprise cosmetic auxiliaries such as are usually used in such preparations, for example preservatives, bactericides, perfumes, antifoams, dyes, pigments which have a coloring action, thickeners, surfactants, emulsifiers, emollients, moisturizers and/or humectants, fats, oils, waxes or other customary constituents of a cosmetic or dermatological preparation, such as alcohols, polyols, polymers, foam stabilizers, electrolytes, organic solvents or silicone derivatives.

In particular, the physiologically compatible sulfinic acids can, according to the invention, also be combined with other antioxidants and/or free-radical scavengers.

According to the invention, favorable antioxidants which may be used are all antioxidants suitable or customary for cosmetic and/or dermatological applications.

The antioxidants are advantageously chosen from the group consisting of amino acids (for example glycine, histidine, tyrosine, tryptophan) and derivatives thereof, imidazoles (for example urocanic acid) and derivatives thereof, peptides such as D,L-carnosine, D-carnosine, L-carnosine and derivatives thereof (for example anserine), carotenoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and furthermore (metal) chelating agents (for example α-hydroxy-fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives (for example ascorbyl palmitate, Mg-ascorbyl palmitate, ascorbyl acetate), tocopherols and derivatives (for example vitamin E acetate), vitamin A and derivatives (vitamin A palmitate) and coniferyl benzoate of benzoic resin, rutic acid and derivatives thereof, butylhydroxytoluene, butylhydroxyanisole, nordihydroguaiacic acid, nordihydroguaiarectic acid, trihydroxybutyrophenone, uric acid and derivatives thereof, mannose and derivatives thereof, sesamol, sesamolin, zinc and derivatives thereof (for example ZnO, ZnSO₄), selenium and derivatives thereof (for example selenium methionine), stilbenes and derivatives thereof (for example stilbene oxide, trans-stilbene oxide) and the derivatives of these active ingredients mentioned which are suitable according to the invention (salts, esters, ethers, sugars, nucleotides, nucleosides, peptides and lipids).

The amount of the abovementioned antioxidants (one or more compounds) in the preparations is preferably from 0.001 to 30% by weight, particularly preferably 0.05-20% by weight, in particular 1-10% by weight, based on the total weight of the formulation.

If vitamin E and/or derivatives thereof is or are the antioxidant or antioxidants, it is advantageous to choose the respective concentrations thereof from the range 0.001-10% by weight, based on the total weight of the formulation.

If vitamin A or vitamin A derivatives or carotenes or derivatives thereof is or are the antioxidant or antioxidants, it is advantageous to choose the respective concentrations thereof from the range 0.001-10% by weight, based on the total weight of the formulation.

Emulsions according to the invention are advantageous and comprise, for example, said fats, oils, waxes and other fatty substances, and also water and an emulsifier, as is customarily used for this type of formulation.

The lipid phase can advantageously be chosen from the following group of substances:

mineral oils, mineral waxes;

oils, such as triglycerides of capric or of caprylic acid, also natural oils such as, for example, castor oil;

fats, waxes and other natural and synthetic fatty substances, preferably esters of fatty acids with alcohols of low C number, for example with isopropanol, propylene glycol or glycerol, or esters of fatty alcohols with alkanoic acids of low C number or with fatty acids;

alkyl benzoates;

silicone oils, such as dimethylpolysiloxanes, diethylpolysiloxanes, diphenylpolysiloxanes and mixed forms thereof.

For the purposes of the present invention, the oil phase of the emulsions, oleogels and hydrodispersions or lipodispersions is advantageously chosen from the group consisting of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, from the group consisting of esters of aromatic carboxylic acids and saturated and/or unsaturated, branched and/or unbranched alcohols having a chain length of from 3 to 30 carbon atoms. Such ester oils can then be advantageously chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of such esters, e.g. jojoba oil.

The oil phase can also advantageously be chosen from the group consisting of branched and unbranched hydrocarbons and hydrocarbon waxes, silicone oils, dialkyl ethers, from the group consisting of saturated or unsaturated, branched or unbranched alcohols, and also fatty acid triglycerides, namely the triglycerol ester of saturated and/or unsaturated, branched and/or unbranched alkanecarboxylic acids having a chain length of from 8 to 24, in particular 12-18, carbon atoms. The fatty acid triglycerides can advantageously be chosen, for example, from the group consisting of synthetic, semi-synthetic and natural oils, e.g. olive oil, sunflower oil, soybean oil, groundnut oil, rapeseed oil, almond oil, palm oil, coconut oil, palm kernel oil and the like.

For the purposes of the present invention, any mixtures of such oil and wax components can also advantageously be used. When required, it may also be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂-C₁₅-alkyl benzoate, caprylic/capric triglyceride and dicaprylyl ether.

Mixtures of C₁₂-C₁₅-alkyl benzoate and 2-ethylhexyl isostearate, mixtures of C₁₂-C₁₅-alkyl benzoate and isotridecyl isononanoate and mixtures of C₁₂-C₁₅-alkyl benzoate, 2-ethylhexyl isostearate and isotridecyl isononanoate are particularly advantageous.

For the purposes of the present invention, of the hydrocarbons, paraffin oil, squalane and squalene can advantageously be used.

The oil phase can advantageously also contain cyclic or linear silicone oils or can consist entirely of such oils, although it is preferable to use an additional content of other oil phase components in addition to the silicone oil or silicone oils.

Cyclomethicone (octamethylcyclotetrasiloxane) is advantageously used as the silicone oil to be used according to the invention. However, other silicone oils can also be advantageously used for the purposes of the present invention, for example hexamethylcyclotrisiloxane, polydimethylsiloxane, poly(methylphenylsiloxane).

Mixtures of cyclomethicone and isotridecyl isononanoate and mixtures of cyclomethicone and 2-ethylhexyl isostearate are also particularly advantageous.

If appropriate, the aqueous phase of the preparations according to the invention advantageously comprises alcohols, diols or polyols of low C number and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, also alcohols of low C number, for example ethanol, isopropanol, 1,2-propanediol and glycerol, and, in particular, one or more thickeners, which can advantageously be chosen from the group consisting of silicon dioxide, aluminum silicates, polysaccharides and derivatives thereof, for example hyaluronic acid, xanthan gum and hydroxypropylmethylcellulose, particularly advantageously from the group consisting of polyacrylates, preferably a polyacrylate from the group consisting of “Carbopols”, for example Carbopols of types 980, 981, 1382, 2984 and 5984, in each case individually or in combination.

In particular, mixtures of the abovementioned solvents are used. In the case of alcoholic solvents, water may be a further constituent.

Emulsions according to the invention are advantageous and comprise, for example, said fats, oils, waxes and other fatty substances, and also water and an emulsifier, as is customarily used for this type of formulation.

Gels according to the invention customarily comprise alcohols of low C number, for example ethanol, isopropanol, 1,2-propanediol, glycerol, and water and/or an abovementioned oil in the presence of a thickener which, in the case of oily-alcoholic gels, is preferably silicon dioxide or an aluminum silicate, and in the case of aqueous-alcoholic or alcoholic gels, is preferably a polyacrylate.

Suitable propellants for preparations according to the invention which can be sprayed from aerosol containers are the customary known readily volatile, liquefied propellants, for example hydrocarbons (propane, butane, isobutane), which may be used alone or in mixtures with one another. Compressed air can also be used advantageously.

Preparations according to the invention can advantageously also comprise substances which absorb UV radiation in the UVB region, the total amount of filter substances being, for example, from 0.1% by weight to 30% by weight, preferably from 0.5 to 10% by weight, in particular from 1.0 to 6.0% by weight, based on the total weight of the preparations, in order to provide cosmetic formulations which protect the skin or hair from the entire range of ultraviolet radiation. They can also be used as sunscreen compositions for hair or skin.

If the preparations according to the invention comprise UVB filter substances, these may be oil-soluble or water-soluble. Advantageous oil-soluble UVB filter substances are, for example:

3-benzylidenecamphor derivatives, preferably 3-(4-methylbenzylidene)camphor and 3-benzylidenecamphor;

4-aminobenzoic acid derivatives, preferably 2-ethylhexyl 4-(dimethylamino)benzoate and amyl 4-(dimethylamino)benzoate;

esters of cinnamic acid, preferably 2-ethylhexyl 4-methoxycinnamate and isopentyl 4-methoxycinnamate;

esters of salicylic acid, preferably 2-ethylhexyl salicylate, 4-isopropylbenzyl salicylate and homomenthyl salicylate,

derivatives of benzophenone, preferably 2-hydroxy-4-methoxybenzophenone, 2-hydroxy-4-methoxy-4′-methylbenzophenone and 2,2′-dihydroxy-4-methoxybenzophenone;

esters of benzalmalonic acid, preferably di(2-ethylhexyl) 4-methoxybenzalmalonate and

2,4,6-Trianilino (P-carbo-2′-ethyl-1′-hexyloxy)-1,3,5-triazine

Advantageous water-soluble UVB filters are, for example:

salts of 2-phenylbenzimidazole-5-sulfonic acid, such as its sodium, potassium or its triethanolammonium salt, and the sulfonic acid itself;

sulfonic acid derivatives of benzophenones, preferably 2-hydroxy-4-methoxybenzophenone-5-sulfonic acid and its salts;

sulphonic acid derivatives of 3-benzylidenecamphor, such as, for example 4-(2-oxo-3-bornylidenemethyl)benzenesulfonic acid, 2-methyl-5-(2-oxo-3-bornylidenemethyl)sulfonic acid and its salts, and also 1,4-di(2-oxo-10-sulfo-3-bornylidenemethyl)benzene and its salts (the corresponding 10-sulfato compounds, for example the corresponding sodium, potassium or triethanolammonium salt) also referred to as benzene-1,4-di(2-oxo-3-bornylidenemethyl)-10-sulfonic acid.

The list of said UVB filters, which can be used in combination with the active ingredient combinations according to the invention, is not, of course, intended to be limiting.

The invention also relates to the use of a combination of one or more physiologically compatible sulfinic acids with at least one UVB filter as antioxidant and to the use of a combination of one or more physiologically compatible sulfinic acids with at least one UVB filter as antioxidant in a cosmetic or dermatological preparation.

It can also be advantageous to combine physiologically compatible sulfinic acids with UVA filters, which have to date customarily been present in cosmetic preparations. These substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. These combinations and preparations comprising these combinations are also provided by the invention. The amounts used may be as for the UVB combination.

The invention also relates to the use of a combination of physiologically compatible sulfinic acids with at least one UVA filter as an antioxidant and to the use of a combination of the active ingredient combinations according to the invention with at least one UVA filter as an antioxidant in a cosmetic or dermatological preparation.

Cosmetic and dermatological preparations with an effective content of physiologically compatible sulfinic acids can also contain inorganic pigments which are normally used in cosmetics for protecting the skin against UV rays. These are oxides of titanium, zinc, zirconium, silicon, manganese, cerium and mixtures thereof, and modifications in which the oxides are the active agents. Particular preference is given to pigments based on titanium dioxide.

These combinations of UVA filter and pigment and preparations which comprise this combination are also provided by the invention. The quantities used may be as stated for the aforementioned combinations.

The cosmetic and dermatological preparations for protecting the hair against UV rays according to the invention are, for example, shampoos, preparations which are applied to the hair when rinsing the hair before or after shampooing, before or after permanent waving, before or after coloring or bleaching, preparations for blow-drying or setting the hair, preparations for coloring or bleaching, a styling and treatment lotion, a hairspray or a permanent wave solution.

The cosmetic and dermatological preparations comprise active ingredients and auxiliaries which are usually used for this type of preparation for hair care and hair treatment. Auxiliaries include preservatives, surfactants, antifoams, thickeners, emulsifiers, fats, oils, waxes, organic solvents, bactericides, perfumes, dyes or pigments whose task is to color the hair or the cosmetic or dermatological preparation itself, electrolytes and substances to combat hair greasiness.

For the purposes of the present invention, electrolytes are taken to mean water-soluble alkali metal, ammonium, alkaline earth metal (including magnesium) and zinc salts of inorganic anions and any mixtures of such salts, it being necessary to ensure that these salts are pharmaceutically or cosmetically safe.

The anions according to the invention are preferably chosen from the group consisting of chlorides, sulfates and hydrogensulfates, phosphates, hydrogenphosphates and linear and cyclic oligophosphates and carbonates and hydrogencarbonates.

Cosmetic preparations which are in the form of a skin cleanser or shampoo preferably comprise at least one anionic, nonionic or amphoteric surfactant, or also mixtures of such substances, sulfinic acids in aqueous medium, and auxiliaries usually used for this purpose. The surfactant and the mixtures of these substances can be present in the shampoo in a concentration of between 1% by weight and 50% by weight.

If the cosmetic or dermatological preparations are in the form of a lotion which is rinsed out and applied, for example, before or after bleaching, before or after shampooing, between two shampooing steps, before or after permanent waving, they are for example, aqueous or aqueous-alcoholic solutions optionally comprising surfactants which can be present in a concentration of between 0.1 and 10% by weight, preferably between 0.2 and 5% by weight.

These cosmetic or dermatological preparations can also be in the form of aerosols with the auxiliaries usually used for this purpose.

A cosmetic preparation in the form of a lotion which is not rinsed out, in particular a lotion for setting the hair, a lotion which is used for blow drying the hair, a styling and treatment lotion, is generally in the form of an aqueous, alcoholic or aqueous-alcoholic solution, and contains at least one cationic, anionic, nonionic or amphoteric polymer or also mixtures thereof, and also sulfinic acids in an effective concentration. The amount of polymers used is, for example, between 0.1 and 10% by weight, preferably between 0.1 and 3% by weight.

Cosmetic preparations for treating and caring for the hair which contain physiologically compatible sulfinic acids can be in the form of emulsions which are of the nonionic or anionic type. Nonionic emulsions comprise, in addition to water, oils or fatty alcohols which may, for example, also be polyethoxylated or polypropoxylated, or also mixtures of the two organic components. These emulsions optionally contain cationic surfactants.

According to the invention, cosmetic preparations for treating and caring for the hair can be in the form of gels which, in addition to an effective content of physiologically compatible sulfinic acids and solvents usually used therefor, preferably water, also contain organic thickeners, e.g. gum arabic, xanthan gum, sodium alginate, cellulose derivatives, preferably methylcellulose, hydroxymethylcellulose, hydroxyethylcellulose, hydroxypropylcellulose, hydroxypropylmethylcellulose or inorganic thickeners, for example aluminum silicates such as, for example, bentonites, or a mixture of polyethylene glycol and polyethylene glycol stearate or distearate. The thickener is present in the gel, for example, in an amount between 0.1 and 30% by weight, preferably between 0.5 and 15% by weight.

The amount of physiologically compatible sulfinic acids in a composition intended for hair is preferably from 0.05% by weight to 10% by weight, in particular from 0.5% by weight to 5% by weight, based on the total weight of the composition.

Aqueous cosmetic cleansers according to the invention or low-water or water-free cleanser concentrates intended for aqueous cleansing may contain anionic, nonionic and/or amphoteric surfactants, for example

conventional soaps, e.g. fatty acid salts of sodium

alkyl sulfates, alkyl ether sulfates, alkanesulfonates and alkylbenzenesulfonates

sulfoacetates

sulfobetaines

sarcosinates

amidosulfobetaines

sulfosuccinates

sulfosuccinic acid monoesters

alkyl ether carboxylates

protein-fatty acid condensates

alkylbetaines and amidobetaines

fatty acid alkanolamides

polyglycol ether derivatives

Cosmetic preparations which are cosmetic skin cleansing preparations can be in liquid or solid form. In addition to physiologically compatible sulfinic acids they preferably contain at least one anionic, nonionic or amphoteric surfactant or mixtures thereof, if desired one or more electrolytes and auxiliaries usually used for this purpose. The surfactant can be present in the cleansing preparations in a concentration of between 1 and 94% by weight, based on the total weight of the preparations.

Cosmetic preparations which are a shampoo preferably comprise, in addition to an effective amount of physiologically compatible sulfinic acids, at least one anionic, nonionic or amphoteric surfactant or mixtures thereof, if desired one electrolyte according to the invention and auxiliaries which are usually used for this purpose. The surfactant can be present in the shampoo in a concentration of between 1% by weight and 94% by weight.

The compositions according to the invention comprise, in addition to the aforementioned surfactants, water and, when required, the additives usual in cosmetics, for example perfume, thickeners, dyes, deodorants, antimicrobial substances, refatting agents, complexing agents and sequestering agents, pearlizing agents, plant extracts, vitamins, active ingredients and the like.

The present invention also relates to a cosmetic method of protecting the skin and hair against oxidative or photooxidative processes which comprises applying a cosmetic composition which comprises an effective concentration of physiologically compatible sulfinic acids in a sufficient quantity to the skin or hair.

The present invention likewise also covers a method of protecting cosmetic or dermatological preparations against oxidation or photooxidation, these preparations being, for example, preparations for the treatment and care of hair, in particular hair colorants, hair lacquers, shampoos, color shampoos, and also make-up products such as, for example, nail varnishes, lipsticks, foundations, washing and shower preparations, creams for the treatment or care of skin or all other cosmetic preparations whose constituents may be associated with stability problems because of oxidation or photooxidation during storage, characterized in that the cosmetic preparations have an effective content of physiologically compatible sulfinic acids.

The amount of physiologically compatible sulfinic acids in these preparations is preferably 0.01-10% by weight, preferably 0.05-5% by weight, in particular 0.1-2.0% by weight, based on the total weight of the preparations.

The invention also relates to the process for the preparation of the cosmetics compositions according to the invention, which comprises incorporating active ingredient combinations according to the invention into cosmetic and dermatological formulations in a manner known per se.

The examples below are intended to illustrate the present invention without limiting it. Unless stated otherwise, all quantities, proportions and percentages are based on the weight and the total amount or on the total weight of the preparations.

| | % by wt. |
|---|---|
| Example 1 (O/W cream): | |
| Glyceryl stearate citrate | 3.00 |
| Stearyl alcohol | 5.00 |
| Octyldodecanol | 6.00 |
| Caprylic/capric triglyceride | 3.00 |
| Sodium carbomer | 0.10 |
| Hypotaurine | 0.20 |
| Glycerol | 3.00 |
| Perfume, preservatives, dyes, antioxidants | q.s. |
| Water | ad 100.00 |
| Example 2 (O/W cream): | |
| Glyceryl stearate citrate | 3.60 |
| Sorbitan monostearate | 1.20 |
| Cetylstearyl alcohol | 1.20 |
| Octyldodecanol | 3.00 |
| Caprylic/capric triglyceride | 3.00 |
| Dicaprylyl ether | 3.00 |
| Hypotaurine | 0.20 |
| Na₃HEDTA | 0.20 |
| Xanthan gum | 0.10 |
| Glycerol | 3.00 |
| Perfume, preservatives, dyes, antioxidants | q.s. |
| Water | ad 100.00 |
| Example 3 (O/W cream): | |
| Glyceryl stearatate citrate | 2.40 |
| Sorbitan monostearate | 0.80 |
| Cetylstearyl alcohol | 0.80 |
| Octyldodecanol | 3.00 |
| Caprylic/capric triglyceride | 3.00 |
| Dicaprylyl ether | 3.00 |
| Xanthan gum | 0.10 |
| Citric acid | 0.10 |
| Sodium citrate | 0.20 |
| Hypotaurine | 0.30 |
| Glycerol | 3.00 |
| Perfume, preservatives, dyes, antioxidants | q.s. |
| Water | ad 100.00 |
| Example 4 (O/W cream): | |
| Glyceryl stearate citrate | 4.00 |
| Cetylstearyl alcohol | 1.20 |
| Octyldodecanol | 3.00 |
| Caprylic/capric triglyceride | 3.00 |
| Squalane | 1.00 |
| Jojoba oil | 1.00 |
| Cyclomethicone | 1.00 |
| Dimethicones | 0.50 |
| Paraffinum liquidum | 1.00 |
| Hydrogenated coconut fatty acid glycerides | 2.00 |
| Xanthan gum | 0.10 |
| Hypotaurine | 0.15 |
| Glycerol | 3.00 |
| Perfume, preservatives, dyes, antioxidants | q.s. |
| Water | ad 100.00 |
| Example 5 (O/W lotion): | |
| Glyceryl stearate citrate | 2.00 |
| Stearyl alcohol | 2.00 |
| Octyldodecanol | 2.00 |
| Caprylic/capric triglyceride | 1.00 |
| Sodium carbomer | 0.10 |
| Hypotaurine | 0.20 |
| Glycerol | 3.00 |
| Perfume, preservatives, dyes, antioxidants | q.s. |
| Water | ad 100.00 |
| Example 6 (Sun protection cream): | |
| Glyceryl stearate citrate | 3.50 |
| Cetylstearyl alcohol | 1.00 |
| Octyldodecanol | 3.00 |
| Caprylic/capric triglycerides | 3.00 |
| Dicaprylyl ether | 3.00 |
| Xanthan gum | 0.10 |
| Sodium carbomer | 0.10 |
| Glycerol | 3.00 |
| Hypotaurine | 0.15 |
| Urea | 4.00 |
| Octyl methoxycinnamate | 4.00 |
| Benzophenone-3 | 3.00 |
| Octyl salicylate | 3.00 |
| Perfume, preservatives, dyes, antioxidants | q.s. |
| Water | ad 100.00 |
| Example 7 (Emulsifier gel): | |
| Glyceryl stearate citrate | 3.60 |
| Sorbitan monostearate | 1.20 |
| Cetylstearyl alcohol | 1.20 |
| Xanthan gum | 0.10 |
| Hypotaurine | 0.20 |
| Glycerol | 3.00 |
| Perfume, preservatives, dyes, antioxidants | q.s. |
| Water | ad 100.00 |

1. A cosmetic or dermatological preparation which has a pH of >5 , with a content of physiologically compatible sulfinic acids.

2. The use of physiologically compatible sulfinic acids as antioxidant and/or as free-radical scavenger in cosmetic or dermatological preparations which have a pH of >5.

3. The preparation as claimed in claim 1 or the use as claimed in claim 2, wherein the physiologically compatible thiosulfonic acid or thiosulfonic acids is/are present in an effective content in cosmetic or dermatological preparations.

4. The preparation as claimed in claim 1 or the use as claimed in claim 2, wherein the physiologically compatible thiosulfonic acid or thiosulfonic acids is/are present in cosmetic or topical dermatological preparations in concentrations of 0.01-10% by weight, preferably 0.05-5% by weight, in particular 0.1-2.0-% by weight, based on the total weight of the preparations.

5. The preparation as claimed in claim 1 or the use as claimed in claim 2, wherein the physiologically compatible thiosulfonic acid chosen is hypotaurine.
